(19) United States
(12) Patent Application Publication
(10) Pub. No.: US 2013/0340327 A1
(43) Pub. Date: Dec. 26, 2013

(54) Process For Preparing Unsaturated Compounds

(71) Applicant: BASF SE, Ludwigshafen (DE)

(72) Inventors: Dominik Ohlmann, Namborn-Roschberg (DE); Lukas J. Gooßen, Kaiserslautern (DE); Markus Dierker, Dusseldorf (DE)

(73) Assignee: BASF SE, Ludwigshafen (DE)

(21) Appl. No.: 13/926,551

(22) Filed: Jun. 25, 2013

Related U.S. Application Data
(60) Provisional application No. 61/664,189, filed on Jun. 26, 2012.

(51) Int. Cl. C07C 67/475

(52) U.S. Cl. CPC C07C 67/475
USPC 44/398

(57) ABSTRACT

Aspects of the present invention relate to processes for preparing compositions comprising unsaturated compounds, wherein (A) one or more unsaturated monocarboxylic acids having 10-24 carbon atoms or esters of these monocarboxylic acids and (B) one or more unsaturated dicarboxylic acids having 4 to 20 carbon atoms or esters thereof are subjected to a tandem isomerization/metathesis reaction in the presence of a palladium catalyst and of a ruthenium catalyst, with the proviso that the palladium catalysts used are compounds which contain at least one structural element Pd—P(R¹R²R³) where the R¹-R³ radicals each independently have 2-10 carbon atoms, each of which may be aliphatic, alicyclic, aromatic or heterocyclic, with the proviso at least one of the R¹-R³ radicals contains a beta-hydrogen, the palladium catalyst being used as such or generated in situ, with the proviso the process is performed in the absence of substances having a pKa of 3 or less.

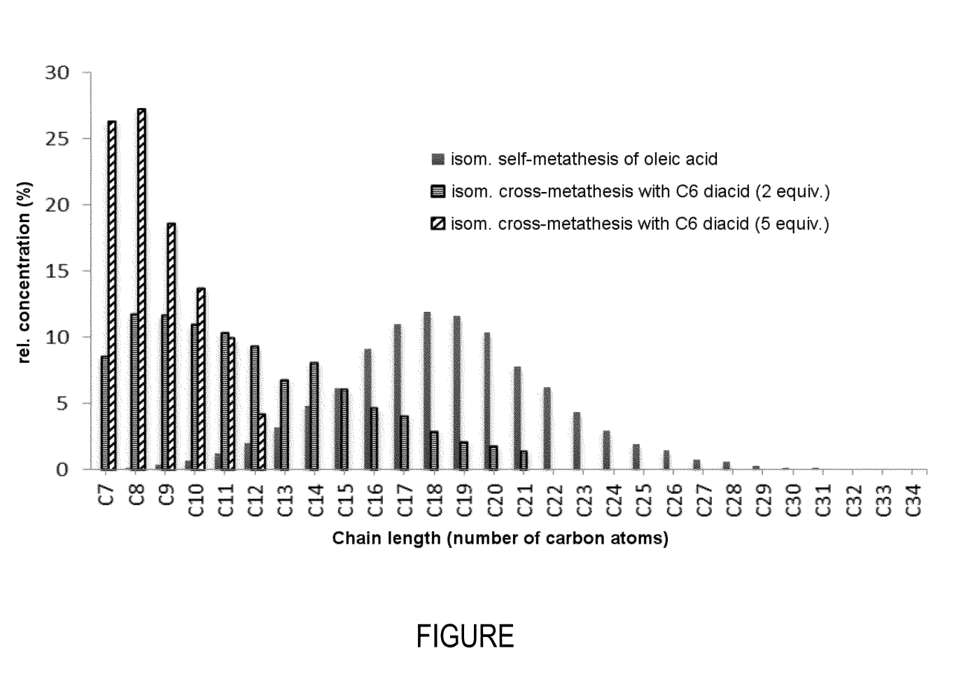

### CROSS-REFERENCE TO RELATED APPLICATIONS

This applications claims priority to U.S. Provisional Application No. 61/664,189, filed Jun. 26, 2012, the entire contents of which are herein incorporated by reference.

### FIELD

Aspects of the present invention relate to one or more processes for preparing unsaturated compounds. These process comprise of a tandem isomerization/metathesis wherein a catalyst system comprising firstly a specific palladium catalyst and secondly a ruthenium catalyst is used.

### BACKGROUND

Processes for preparing unsaturated alpha,omega-dicarboxylic diesters proceeding from unsaturated carboxylic acids are described in the prior art. Ngo et al. (JAOCS, Vol. 83, No. 7, p. 629-634, 2006) describes the metathesis of unsaturated carboxylic acids with the aid of first and second generation Grubbs catalysts.

WO 2010/020368 describes a process for preparing unsaturated alpha,omega-dicarboxylic acids and alpha,omega-dicarboxylic diesters, in which unsaturated carboxylic acids and/or esters of unsaturated carboxylic acids are converted in the presence of two specific ruthenium catalysts.

### SUMMARY

One aspect of the invention pertains to a method of preparing compositions comprising unsaturated compounds. In one or more embodiments, the method comprises subjecting:
(A) one or more unsaturated monocarboxylic acids having 10 to 24 carbon atoms or esters of these monocarboxylic acids and
(B) one or more unsaturated dicarboxylic acids having 4 to 20 carbon atoms or esters thereof
to a tandem isomerization/metathesis reaction in the presence of a palladium catalyst and of a ruthenium catalyst,
with the proviso that the palladium catalysts used are compounds which contain at least one structural element Pd—P(R¹R²R³) where the R¹ to R³ radicals each independently have 2 to 10 carbon atoms, each of which may be aliphatic, alicyclic, aromatic or heterocyclic,
and with the proviso that at least one of the R¹ to R³ radicals contains a beta-hydrogen, the palladium catalyst being used as such or generated in situ, with the proviso that the process is performed in the absence of substances having a pKa of 3 or less.

In one or more embodiments, the palladium catalyst contains two palladium atoms per molecule. In further embodiments, the two palladium atoms are joined to one another via a spacer X, the spacer being selected from halogen, oxygen, O-alkyl, sulfur, sulfur-alkyl, disubstituted nitrogen, carbon monoxide, nitrile, and diolefin. In some embodiments, the palladium catalysts have a structure represented by formula (I)

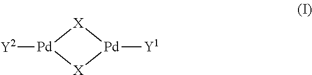

in which: X is a spacer selected from halogen, oxygen and O-alkyl, Y¹ is a P(R¹R²R³) group in which R¹, R² and R³ are each as defined above, Y² is a P(R⁴R⁵R⁶) group in which R⁴, R⁵ and R⁶ each independently have 2 to 10 carbon atoms, each of which may be aliphatic, alicyclic, aromatic or heterocyclic.

In one or more embodiments, the palladium catalysts have a structure represented by formula (I-a)

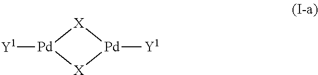

in which: X is a spacer selected from halogen, oxygen and O-alkyl, Y¹ is a P(R¹R²R³) group in which R¹, R² and R³ are each as defined above, Y² is a P(R⁴R⁵R⁶) group in which R⁴, R⁵ and R⁶ each independently have 2 to 10 carbon atoms, each of which may be aliphatic, alicyclic, aromatic or heterocyclic. In some embodiments, the palladium catalysts have a structure represented by the formula (I-a)

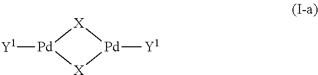

in which the spacer X is bromine and the R¹, R² and R³ radicals are each defined as tert-butyl. In one or more embodiments, the palladium catalyst is a homogeneous or heterogeneous catalyst. In some embodiments, the compounds (B) comprise unsaturated dicarboxylic acids and dicarboxylic acid derivatives having 4 to 10 carbon atoms.

In some embodiments, the reaction is carried out in an aprotic solvent. In one or more embodiments, the reaction is carried out in the absence of a solvent. In some embodiments, the reaction is carried out in the absence of acids having a pKa of 3 or less. In one or more embodiments, the reaction is carried out in the absence of oxygen. In some embodiments, the compounds (A) comprise a compound selected from the group consisting of the unsaturated carboxylic acids having 14 to 24 carbon atoms and the esters of unsaturated carboxylic acids having 14 to 24 carbon atoms. In one or more embodiments, the reaction is carried out at a temperature of about 40 to about 80° C.

### BRIEF DESCRIPTION OF FIGURE

The FIGURE is a graph showing the relative concentration of several compounds having various chain lengths produced by isomerization self-metathesis of oleic acid, and isomerization cross-metathesis with 2 equivalents of a C6 diacid and 5 equivalents of a C6 diacid.

### DETAILED DESCRIPTION

One or more aspects of the present invention provides a novel process which enables the production of unsaturated compounds (which is understood to mean compounds with C═C double bonds) from unsaturated monocarboxylic acids and esters of such monocarboxylic acids.

Accordingly, one aspect of the present invention provides a process for preparing compositions comprising unsaturated compounds, wherein (A) one or more unsaturated monocarboxylic acids having 10 to 24 carbon atoms or esters of these monocarboxylic acids and (B) one or more unsaturated dicarboxylic acids having 4 to 20 carbon atoms or esters thereof are subjected to a tandem isomerization/metathesis reaction in the presence of a palladium catalyst and of a ruthenium catalyst, with the proviso that the palladium catalysts used are compounds which contain at least one structural element Pd—P(R¹R²R³) where the R¹ to R³ radicals each independently have 2 to 10 carbon atoms, each of which may be aliphatic, alicyclic, aromatic or heterocyclic, with the proviso that at least one of the R¹ to R³ radicals contains a beta-hydrogen, the palladium catalyst being used as such or generated in situ, with the proviso that the process is performed in the absence of substances having a pKa of 3 or less.

In one or more embodiments, the process according to the invention is a tandem isomerization/metathesis and has numerous advantages:
The catalysts do not need any chemical activation of any kind. More particularly, the palladium catalyst, which brings about a C═C isomerization before and after the metathesis step, works by itself without needing to be activated, for instance, by protic solvents and/or strong acids (which is understood here to mean acids having a pKa of 3 or less).
Only moderate amounts of the bimetallic Pd/Ru catalyst system are required.
The catalyst system is such that the Pd catalysts (which bring about a C═C isomerization) and Ru catalysts (which bring about metathesis) present therein do not impair or inhibit the effects of each other, which is not a given.
The catalyst system does not only work when the compounds (A) used are compounds having exclusively one C═C double bond, but it is also possible for several C═C double bonds to be present. This too is not a given. In operating practice, this is a great advantage, for instance, when the compound (A) used is oleic acid. In that case, it is not necessary that this oleic acid has an extremely high purity; instead, it is also possible to use oleic acid of technical-grade quality, for instance one containing approximately 80% oleic acid and 20% linoleic acid.
The catalyst system suppresses the formation of lactones (which are to be expected per se when, for example, unsaturated fatty acids such as oleic acid are admixed with an isomerization catalyst.
The process according to one or more embodiments of the invention can be performed without solvent if desired.
The process according to some embodiments of the invention requires only very mild temperatures.

### The Reactants

### The Reactants (A)

The compounds (A) may be unsaturated monocarboxylic acids having 10 to 24 carbon atoms or esters of these monocarboxylic acids. The monocarboxylic acids may optionally be branched. The C═C double bonds of the monocarboxylic acids may be present either in cis or in trans configuration. It is possible for one or more C═C double bonds to be present.

The unsaturated monocarboxylic acids (A) used are preferably compounds of the formula R¹—COOH where the R¹ radical comprises 9 to 23 carbon atoms. The R¹ radical may be cyclic or acyclic (noncyclic); the R¹ radical is preferably acyclic, and it may be branched or unbranched. Monocarboxylic acids having an unbranched R¹ radical are preferred.

The following brief notation is used to describe the unsaturated monocarboxylic acids: the first number describes the total number of carbon atoms in the monocarboxylic acids, the second number the number of double bonds and the number in brackets the position of the double bond in relation to the carboxyl group. Thus, the brief notation for oleic acid is 18:1 (9). When the double bond is in the trans configuration, this is represented by the abbreviation “tr”. Thus, the brief notation for elaidic acid is 18:1 (tr9).

Suitable monounsaturated monocarboxylic acids include, for example, myristoleic acid [14:1 (9), (9Z)-tetradeca-9-enoic acid], palmitoleic acid [16:1 (9); (9Z)-hexadeca-9-enoic acid], petroselic acid [(6Z)-octadeca-6-enoic acid], oleic acid [18:1 (9); (9Z)-octadeca-9-enoic acid], elaidic acid [18:1 (tr9); (9E)-octadeca-9-enoic acid)], vaccenic acid [18:1 (tr11); (11E)-octadeca-11-enoic acid], gadoleic acid [20:1 (9); (9Z)-eicosa-9-enoic acid], eicosenoic acid (=gondoic acid) [20:1 (11); (11Z)-eicosa-11-enoic acid], cetoleic acid [22:1 (11); (11Z)-docosa-11-enoic acid], erucic acid [22:1 (13); (13Z)-docosa-13-enoic acid], nervonic acid [24:1 (15); (15Z)-tetracosa-15-enoic acid]. Also suitable are functionalized monounsaturated monocarboxylic acids, for instance ricinoleic acid, furan fatty acids, methoxy fatty acids, keto fatty acids and epoxy fatty acids such as vernolic acid (cis-12,13-epoxyoctadec-cis-9-enoic acid), and finally also branched monocarboxylic acids such as phytanoic acid.

Suitable polyunsaturated monocarboxylic acids include, for example, linoleic acids [18:2 (9,12); (9Z,12Z)-octadeca-9,12-dienoic acid], alpha-linolenic acid [18:3 (9,12,15); (9Z,12Z,15Z)-octadeca-9,12,15-trienoic acid], gamma-linolenic acid [18:3 (6,9,12); (6Z,9Z,12Z)-octadeca-6,9,12-trienoic acid], calendic acid [18:3 (8,10,12); (8E,10E,12Z)-octadeca-8,10,12-trienoic acid], punicic acid [18:3 (9,11,13); (9Z,11E,13Z)-octadeca-9,11,13-trienoic acid], alpha-eleostearic acid [18:3 (9,11,13); (9Z,11E,13E)-octadeca-9,11,13-trienoic acid], arachidonic acid [20:4 (5,8,11,14), (5Z,8Z,11Z,14Z)-eicosa-5,8,11,14-tetraenoic acid], timnodonic acid [20:5 (5,8,11,14,17), (5Z,8Z,11Z,14Z,17Z)-eicosa-5,8,11,14,17-pentaenoic acid], clupanodonic acid [22:5 (7,10,13,16,19), (7Z,10Z,13Z,16Z,19Z)-docosa-7,10,13,16,19-pentaenoic acid], cervonic acid [22:6 (4,7,10,13,16,19), (4Z,7Z,10Z,13Z,16Z,19Z)-docosa-4,7,10,13,16,19-hexaenoic acid].

Suitable reactants (A) additionally may include esters of the mono- or polyunsaturated monocarboxylic acids mentioned. In some embodiments, suitable esters are especially esters of these monocarboxylic acids with alcohols R²—OH where R² is an alkyl radical having 1 to 8 carbon atoms. Examples of suitable R² radicals include: methyl, ethyl, propyl, isopropyl, butyl, 2-methylpropyl, pentyl, 2,2-dimethylpropyl, 2-methylbutyl, 3-methylbutyl, hexyl, 1-methylpentyl, 2-methylpentyl, 3-methylpentyl, 4-methylpentyl, 1-ethylbutyl, 2-ethylbutyl, heptyl and octyl radicals.

Suitable reactants (A) are additionally esters of the mono- or polyunsaturated monocarboxylic acids mentioned with glycerol (=glyceryl esters). In this case, glyceryl monoesters (=monoglycerides, monoacylglycerol), glyceryl diesters (=diglycerides, diacylglycerol) and glyceryl triesters (=triglycerides, triacylglycerol), and also mixtures of these different glyceryl esters, are suitable.

In one or more embodiments, the unsaturated monocarboxylic acids or the esters of the unsaturated monocarboxylic acids may be present either individually or in mixtures with one another. While in some embodiments, exclusively one unsaturated monocarboxylic acid or the ester of only one unsaturated monocarboxylic acid is used, the reaction which takes place in the context of the process according to the invention is one which can be classified as an isomerizing self-metathesis. When different unsaturated monocarboxylic acids or esters of different unsaturated monocarboxylic acids are used, the reaction which takes place in the context of the process according to the invention is one which can be classified as an isomerizing cross-metathesis.

In a particular embodiment of the invention, monounsaturated monocarboxylic acids and/or esters of monounsaturated monocarboxylic acids and/or mixtures of the monounsaturated monocarboxylic acids or mixtures of the esters of monounsaturated monocarboxylic acids are used.

### The Reactants (B)

In one or more embodiments of the invention, the reactants (B) used in the process according to the invention are unsaturated dicarboxylic acids having 4 to 20 carbon atoms and contain at least one C═C double bond per molecule. The reactants (B) may contain 4 to 10 carbon atoms per molecule. The dicarboxylic acids may be straight-chain or branched. The C═C double bonds may be present both in cis- and in trans-configuration.

The compounds (B) may contain further functional groups which are inert under the reaction conditions. Examples of such functional groups are, for instance, COOR, OH, OR, C═C, halogen and CN, where R is an alkyl group.

Examples of suitable reactants (B) are especially unsaturated dicarboxylic acids and dicarboxylic acid derivatives having 4 to 10 carbon atoms, preferably 4 to 8 carbon atoms, especially 4 to 6 carbon atoms. Examples of these are maleic acid and esters thereof and (E)-3-hexenedicarboxylic acid and esters thereof.

Since, in the context of the process according to the invention, one or more reactants (B) are also used in addition to the reactants (A), the metathesis step of the tandem isomerization/metathesis reaction is a cross-metathesis.

It has been found that the cross-metathesis of oleic acid with short-chain dicarboxylic acids leads to the formation of mono- and dicarboxylic acids having principally moderate chain length. In the case of maleic acid, the cross-metathesis rate was much lower than the self-metathesis rate of oleic acid. It has been found, however, that, in the case of (E)-3-hexenedicarboxylic acid, the cross-metathesis proceeded at the same rate as the self-metathesis. The product distribution suggested the following: both the oleic acid and the (E)-3-hexenedicarboxylic acid were converted quantitatively. The mean chain length of all product fractions was essentially lower than the self-metathesis rate of oleic acid, and the dicarboxylic acid fraction became dominant. In this regard, reference is made to the FIGURE.

If, in the context of the process according to the invention, one or more reactants (B) are also used in addition to the reactants (A), the molar ratio of (A):(B) is preferably set to a value in the range from 1:0.05 to 1:5.

### The Catalysts

In one or more embodiments, the process according to the invention is performed in the presence in the presence of a specific palladium catalyst and a ruthenium catalyst.

### The Palladium Catalyst

In some embodiments, the palladium catalysts used are compounds which contain at least one structural element Pd—P(R¹R²R³) where the R¹ to R³ radicals each independently have 2 to 10 carbon atoms, each of which may be aliphatic, alicyclic, aromatic or heterocyclic, with the proviso that at least one of the R¹ to R³ radicals contains a beta-hydrogen, the palladium catalyst being used as such or generated in situ.

Aliphatic radicals may be linear or branched; they may also be in cyclic form; the structural elements mentioned may also be present in combination. Aromatic radicals may also have alkyl substituents. A beta-hydrogen is present when the Pd—P—C≡C—H arrangement is present in the palladium catalyst.

As explained above, the palladium catalyst may be used as such or generated in situ.

It is explicitly emphasized that the palladium catalysts for use in accordance with the invention work by themselves, which is understood to mean that they do not require a chemical activation by an additional activating substance.

The palladium catalysts may be mono- or polynuclear.

In one or more embodiments, palladium catalysts containing two palladium atoms per molecule are used.

In some embodiments, palladium catalysts containing two palladium atoms per molecule are used, where the two palladium atoms are joined to one another via a spacer X.

Therefore, these palladium catalysts contain the structural element Pd—X—Pd.

The nature of the spacer is not subject to any restriction per se. Suitable spacers X are, for example, halogen, oxygen, O-alkyl, sulfur, sulfur-alkyl, disubstituted nitrogen, carbon monoxide, nitrile, diolefin.

In a preferred embodiment, the palladium catalysts used are the compounds (I)

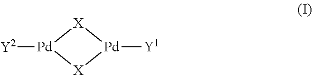

in which: X is a spacer selected from halogen, oxygen and O-alkyl, Y¹ is a P(R¹R²R³) group in which R¹, R² and R³ are each as defined above, Y² is a P(R⁴R⁵R⁶) group in which R⁴, R⁵ and R⁶ each independently have 2 to 10 carbon atoms, each of which may be aliphatic, alicyclic, aromatic or heterocyclic.

It follows from this definition that the compounds (I) can contain at least one beta-hydrogen in the structural element Pd—Y1 (owing to the R¹ to R³ radicals present therein). In the structural element Pd—Y2, a beta-hydrogen need not necessarily be present.

In one or more embodiments, particular preference is given to those compounds (I) in which the spacer is halogen and especially bromine. In further embodiments, very particular preference is given to those compounds (I) in which the spacer is bromine and the R¹, R² and R³ radicals are each defined as tert-butyl.

In some embodiments, the palladium catalysts used are the compounds (I-a)

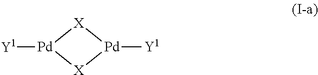

in which: X is a spacer selected from halogen, oxygen and O-alkyl, Y¹ is a P(R¹R²R³) group in which R¹, R² and R³ are each as defined above, Y² is a P(R⁴R⁵R⁶) group in which R⁴, R⁵ and R⁶ each independently have 2 to 10 carbon atoms, each of which may be aliphatic, alicyclic, aromatic or heterocyclic.

In some embodiments, particular preference is given to those compounds (I-a) in which the spacer is halogen and especially bromine.

In further embodiments, very particular preference is given to those compounds (I-a) in which the spacer is bromine and the R¹, R² and R³ radicals are each defined as tert-butyl.

As already explained, the palladium catalyst may be used as such or generated in situ. In situ generation can mean, for example, for a palladium catalyst of the (I) or (I-a) type, that a compound L₃-Pd—X—Pd-L₃ where L represents phosphine ligands without beta-hydrogen is used and converted by ligand exchange in situ to a compound (I) or (I-a).

In one or more embodiments, the palladium catalyst is a homogeneous catalyst.

In some embodiments, the palladium catalyst is a heterogeneous catalyst. In a specific embodiment, a palladium catalyst of the formula (I) is immobilized via the Y¹ and/or Y² group on a solid substrate or in an ionic liquid.

The palladium catalyst may be used in an amount in the range from 0.01 to 2.0 mol %-based on the amount of reactant (A) used; the range from 0.1 to 1.0 mol % is particularly preferred.

### The Ruthenium Catalyst

The chemical nature of the ruthenium catalyst is not critical per se.

Examples of suitable ruthenium catalysts include:

Catalyst (II-a) with the following structural formula:

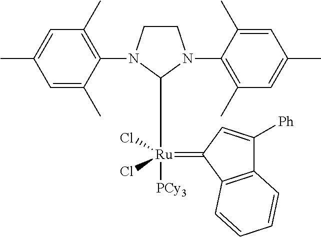

The chemical name for this catalyst is dichloro[1,3-bis(mesityl)-2-imidazolidinylidene]-(3-phenyl-1H-inden-1-ylidene)(tricyclohexylphosphine)ruthenium(II), CAS No. 536724-67-1. It is commercially available under the Neolyst™ M2 name from Umicore.

Catalyst (II-b) with the following structural formula:

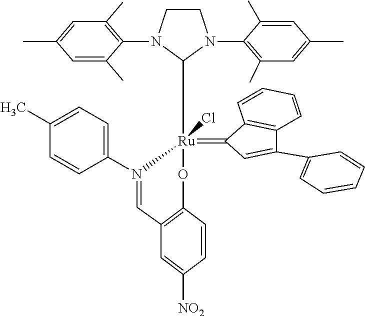

The chemical name for this catalyst is [1,3-bis(2,4,6-trimethylphenyl)-2-imidazolidinylidene]-[2-[[(4-methylphenyl)imino]methyl]-4-nitrophenolyl]-[3-phenyl-1H-inden-1-ylidene]ruthenium(II) chloride, CAS No. 934538-04-2. It is commercially available, for example, under the Neolyst™ M41 name from Umicore.

Catalyst (II-c) with the following structural formula:

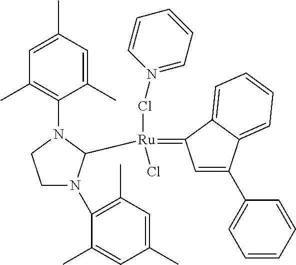

The chemical name for this catalyst is 3-bis(mesityl)-2-imidazolidinylidene]-[2-[[2-methylphenyl)imino]methyl]phenolyl]-[3-phenyl-1H-inden-1-ylidene]-ruthenium(II) chloride. CAS No. 1031262-76-6. It is commercially available, for example, under the Neolyst™ M31 name.

Catalyst (II-d) with the following structural formula:

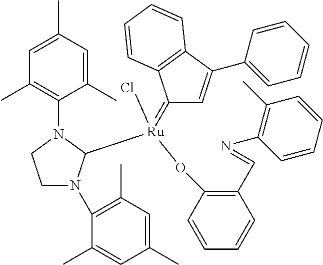

The chemical name for this catalyst is 3-bis(mesityl)-2-imidazolidinylidene]-[2-[[(2-methylphenyl)imino]methyl]phenolyl]-[3-phenyl-1H-inden-1-ylidene]-ruthenium(II) chloride. CAS No. 934538-12-2. It is commercially available, for example, under the Neolyst™ M42 name from Umicore.

Catalyst (II-e) with the following structural formula:

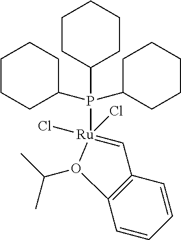

The chemical name for this catalyst is dichloro(o-isopropoxyphenylmethylene)(tricyclohexylphosphine)ruthenium(II). CAS No. 203714-71-0. It is commercially available, for example, under the first generation Hoveyda-Grubbs catalyst name.

The ruthenium catalyst is preferably used in an amount in the range from 0.01 to 5 mol %-based on the amount of reactant (A) used; the range from 0.3 to 1.5 mol % is particularly preferred.

### Reaction Conditions

### Temperature

In one or more embodiments, the process according to the invention is performed at temperatures in the range from about 25 to about 90° C., and especially about 40 to about 80° C. In further embodiments, the temperature ranges from about 50 to about 70° C.

### Solvent

The process can be performed in customary organic solvents in which the reactants (A) or the reactants (A) and (B) and the catalysts used—if the catalysts are used in the form of homogeneous catalysts—dissolve.

It should be stated explicitly that the compounds covered by the definition of the reactants (A) and (B) are not solvents in the context of the present invention, which means that solvents must be structurally different from the compounds (A) and (B).

In some embodiments, the solvent comprises one or more aprotic solvents, for instance hydrocarbons (e.g. hexane or tetrahydrofuran).

In one or more embodiments of the invention, the process is performed without solvent.

### Other Parameters

In some embodiments, the reaction may be performed in the absence of acids having a pKa of 3 or less. Examples of acids having a pKa of 3 or less are, for instance, mineral acids, p-toluenesulfonic acid, methanesulfonic acid.

In some embodiments, the process is carried out in the absence of oxygen, for example in an inert gas stream (for example under nitrogen or argon or by means of passage of nitrogen or argon), or under reduced pressure. If desired, it is also possible for component (B) itself, if it is used and is present in the gaseous state under the reaction conditions, to serve as the inert gas.

### Purification

According to one or more embodiments of the process according to the invention, which is an isomerizing metathesis (=a tandem isomerization/metathesis reaction), leads to substance mixtures whose complexity can be controlled by process parameters including the molar ratio of reactants A and B, nature of reactant B, partial pressure of a gaseous reactant B, reaction regime under reduced pressure, molar ratio of the catalysts, reaction time and temperature. If desired, the substance mixtures can be subjected to a separation by customary processes, for example by distillation, by fractional crystallization or by extraction.

It is optionally possible to subject products obtained by the process according to the invention to a hydrogenation or another cross-metathesis. The latter (another cross-metathesis) may be a desired option if conversion of the monocarboxylates present in the product mixture to dicarboxylates is desired.

### Use

The mixtures of olefins, mono- and dicarboxylates obtainable in accordance with one or more embodiments of the invention from unsaturated fatty acids are similar as such to the fuel used under the name “metathesized biodiesel”, but can if desired also be fractionated into a monoester fraction and a diester fraction, each of which have their own possible uses: monocarboxylate mixtures are suitable, for instance, for plastics applications, surfactants, hydraulic oils and lubricants. In some embodiments, unsaturated dicarboxylates cannot be obtained from mineral oil but play an important role for the production of odorants, adhesives and specialty antibiotics. At the same time, due to the double bond present, they enable further modifications, for example for novel biobased polyesters, polyamides, polyurethanes, resins, fibers, coatings and adhesives.

### EXAMPLES

Catalysts used:

(A): Pd dimer (Pd(dba)₂=1,2-bis(di-t-butylphosphinomethyl)benzene

(B): Umicore M4₂

The chemical name of this catalyst is 3-bis(mesityl)-2-imidazolidinylidene]-[2-[[(2-methylphenyl)imino]methyl]phenolyl]-[3-phenyl-1H-inden-1-ylidene]ruthenium(II) chloride.

(C): 2nd Hoveyda-Grubs.

The chemical name of this caltyast is dichloro(o-isopropoxy-phenylmethylene)(tricyclohexylphosphine)ruthenium(II).

### Example 1

### Isomerizing Cross-Metathesis of Oleic Acid and Maleic Acid

A 20 ml reaction vessel with beaded rim and stirrer bar was initially charged with catalyst (A) (5.8 mg, 7.5 μmol, 0.0075 equiv.), catalyst (B) (12.7 mg, 15 μmol, 0.015 equiv.) and maleic acid (234 mg, 2.0 equiv.), and the vessel was closed with a septum and purged three times with argon. THF (3.0 ml) and oleic acid (90%, 314 mg, 1.0 mmol) were added by syringe and the mixture was stirred at 70° C. for 16 h.

After cooling to room temperature, methanol (2 ml) and conc. sulfuric acid (50 μl) were added by syringe and the mixture was stirred at 70° C. for 2 h. The GC of the mixture showed full conversion [>97%] to a mixture of olefins, unsaturated mono- and dimethyl esters, as reported in Table 1.

TABLE 1
Product distribution according to Example 1

| Fraction | Olefins | Monoesters | Diesters |
|---|---|---|---|
| C9 | | 0.2 | |
| C10 | | 0.61 | |
| C11 | | 0.68 | |
| C12 | | 0.63 | |
| C13 | | 1.32 | |
| C14 | | 2.17 | |
| C15 | | 3.13 | 0.46 |
| C16 | 0.68 | 3.05 | 0.56 |
| C17 | 0.86 | 2.95 | 0.94 |
| C18 | 1.92 | 2.82 | 1.21 |
| C19 | 1.33 | 2.01 | 1.19 |
| C20 | | 1.84 | 1.12 |
| C21 | | 1.58 | 1.12 |
| C22 | | 1.22 | 1.02 |
| C23 | | 0.78 | 0.54 |
| C24 | | 0.74 | |
| C25 | | 0.51 | |
| C26 | | 0.5 | |
| C27 | | 0.33 | |
| C28 | | 0.16 | |

^{a}The totality of the double bond isomers of the same chain length is reported in each case.

### Example 2

### Isomerizing Cross-Metathesis of Methyl Oleate and Maleic Acid

A 20 ml reaction vessel with beaded rim and stirrer bar was initially charged with catalyst (A) (6.1 mg, 7.5 μmol, 0.0075 equiv.), catalyst (B) (12.7 mg, 15 μmol, 0.015 equiv.) and maleic acid (348 mg, 3.0 equiv.), and the vessel was closed with a septum and purged three times with argon. Methyl oleate (90% pure, 329 mg, 1.0 mmol) was added by syringe and the mixture was stirred at 70° C. for 16 h. After cooling to room temperature, methanol (2 ml) and conc. sulphuric acid (50 μl) were added by syringe and the mixture was stirred at 70° C. for 2 h.

The GC of the mixture showed full conversion [>97%] to a mixture of olefins, unsaturated mono- and dimethyl esters.

### Example 3

### Isomerizing Cross-Metathesis of Methyl Oleate and Dimethyl Maleate

A 20 ml reaction vessel with beaded rim and stirrer bar was initially charged with catalyst (A) (5.3 mg, 6.5 μmol, 0.0065 equiv.) and catalyst (C) (9.0 mg, 15 μmol, 0.015 equiv.), and the vessel was closed with a septum and purged three times with argon. Methyl oleate (90% pure, 329 mg, 1.0 mmol) and dimethyl maleate (144 mg, 1.0 mmol, 1.0 equiv.) were added by syringe and the mixture was stirred at 70° C. for 16 h. The GC of the mixture showed 88% conversion to a mixture of olefins, unsaturated mono- and dimethyl esters, as reported in table 2.

TABLE 1
Product distribution according to example 2.

| Fraction | Olefins | Monoesters | Diesters |
|---|---|---|---|
| C10 | | 0.43 | |
| C11 | | 0.73 | |
| C12 | | 1.06 | |
| C13 | | 1.71 | |
| C14 | | 1.95 | |
| C15 | | 2.68 | 0.31 |
| C16 | | 2.78 | 0.64 |
| C17 | 0.79 | 2.74 | 0.79 |
| C18 | 1.88 | 2.16 | 1.47 |
| C19 | 1.49 | 2.43 | 1.37 |
| C20 | 1.24 | 1.75 | 1.34 |
| C21 | 0.41 | 1.45 | 0.89 |
| C22 | | 0.92 | 0.43 |
| C23 | | 0.7 | |
| C24 | | 0.26 | |
| C25 | | 0.26 | |

^{a}the totality of the double bond isomers of the same chain length is reported in each case.

### Example 4

### Isomerizing Cross-Metathesis of Methyl Oleate and Maleic Acid

A 20 ml reaction vessel with beaded rim and stirrer bar was initially charged with catalyst (A) (1.5 mg, 1.9 μmol, 0.0075 equiv.), catalyst (B) (3.2 mg, 3.8 μmol, 0.015 equiv.) and maleic acid (58 mg, 0.5 mmol, 2.0 equiv.), and the vessel was closed with a septum and purged three times with argon. THF (1.5 ml) and methyl oleate (90% pure, 82 mg, 0.25 mmol) were added by syringe and the mixture was stirred at 70° C. for 16 h.

After cooling to room temperature, methanol (2 ml) and conc. sulphuric acid (50 μl) were added by syringe and the mixture was stirred at 70° C. for 2 h. The GC of the mixture showed full conversion [>97%] to a mixture of olefins, unsaturated mono- and dimethyl esters.

### Example 5

### Isomerizing Cross-Metathesis of Methyl Oleate and Fumaric Acid

A 20 ml reaction vessel with beaded rim and stirrer bar was initially charged with catalyst (A) (5.1 mg, 6.3 μmol, 0.025 equiv.), catalyst (B) (3.2 mg, 3.8 μmol, 0.015 equiv.) and fumaric acid (88 mg, 0.75 mmol, 3.0 equiv.), and the vessel was closed with a septum and purged three times with argon. Toluene (1.5 ml) and methyl oleate (90% pure, 82 mg, 0.25 mmol) were added by syringe and the mixture was stirred at 70° C. for 16 h.

After cooling to room temperature, methanol (2 ml) and conc. sulphuric acid (50 μl) were added by syringe and the mixture was stirred at 70° C. for 2 h. The GC of the mixture showed full conversion [>97%] to a mixture of olefins, unsaturated mono- and dimethyl esters.

### Example 6

### Isomerizing Cross-Metathesis of Methyl Oleate and Fumaric Acid

A 20 ml reaction vessel with beaded rim and stirrer bar was initially charged with catalyst (A) (5.1 mg, 6.3 μmol, 0.025 equiv.), catalyst (B) (3.2 mg, 3.8 μmol, 0.015 equiv.) and fumaric acid (88 mg, 0.75 mmol, 3.0 equiv.), and the vessel was closed with a septum and purged three times with argon. THF (1.5 ml) and methyl oleate (90% pure, 82 mg, 0.25 mmol) were added by syringe and the mixture was stirred at 70° C. for 16 h.

After cooling to room temperature, methanol (2 ml) and conc. sulphuric acid (50 μl) were added by syringe and the mixture was stirred at 70° C. for 2 h. The GC of the mixture showed full conversion [>97%] to a mixture of olefins, unsaturated mono- and dimethyl esters.

### Example 7

### Isomerizing Cross-Metathesis of Oleic Acid and Maleic Acid

A 20 ml reaction vessel with beaded rim and stirrer bar was initially charged with catalyst (A) (5.8 mg, 7.5 μmol, 0.075 equiv.), catalyst (B) (12.7 mg, 15 μmol, 0.015 equiv.) and maleic acid (234 mg, 2.0 mmol, 2.0 equiv.), and the vessel was closed with a septum and purged three times with argon. Hexane (3.0 ml) and oleic acid (90% pure, 314 mg, 1.0 mmol) were added by syringe and the mixture was stirred at 70° C. for 16 h.

After cooling to room temperature, methanol (2 ml) and conc. sulphuric acid (50 μl) were added by syringe and the mixture was stirred at 70° C. for 2 h. The GC of the mixture showed 85% conversion to a mixture of olefins, unsaturated mono- and dimethyl esters.

1. A method of preparing compositions comprising unsaturated compounds, the method comprising:
subjecting:
(A) one or more unsaturated monocarboxylic acids having 10 to 24 carbon atoms or esters of these monocarboxylic acids and
(B) one or more unsaturated dicarboxylic acids having 4 to 20 carbon atoms or esters thereof
to a tandem isomerization/metathesis reaction in the presence of a palladium catalyst and of a ruthenium catalyst,
with the proviso that the palladium catalysts used are compounds which contain at least one structural element Pd—P(R¹R²R³) where the R¹ to R³ radicals each independently have 2 to 10 carbon atoms, each of which may be aliphatic, alicyclic, aromatic or heterocyclic,
and with the proviso that at least one of the R¹ to R³ radicals contains a beta-hydrogen, the palladium catalyst being used as such or generated in situ, with the proviso that the process is performed in the absence of substances having a pKa of 3 or less.

2. The method of claim 1, wherein the palladium catalyst contains two palladium atoms per molecule.

3. The method of claim 2, wherein the two palladium atoms are joined to one another via a spacer X, the spacer being selected from halogen, oxygen, O-alkyl, sulfur, sulfur-alkyl, disubstituted nitrogen, carbon monoxide, nitrile, and diolefin.

4. The method of claim 1, wherein the palladium catalysts have a structure represented by formula (I) 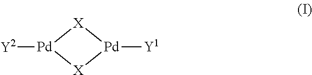
in which: X is a spacer selected from halogen, oxygen and O-alkyl, Y¹ is a P(R¹R²R³) group in which R¹, R² and R³ are each as defined above, Y² is a P(R⁴R⁵R⁶) group in which R⁴, R⁵ and R⁶ each independently have 2 to 10 carbon atoms, each of which may be aliphatic, alicyclic, aromatic or heterocyclic.

5. The method of claim 1, wherein the palladium catalysts have a structure represented by formula (I-a) 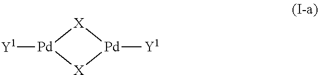
in which: X is a spacer selected from halogen, oxygen and O-alkyl, Y¹ is a P(R¹R²R³) group in which R¹, R² and R³ are each as defined above, Y² is a P(R⁴R⁵R⁶) group in which R⁴, R⁵ and R⁶ each independently have 2 to 10 carbon atoms, each of which may be aliphatic, alicyclic, aromatic or heterocyclic.

6. The method of claim 1, wherein the palladium catalysts have a structure represented by the formula (I-a) 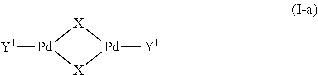
in which the spacer X is bromine and the R¹, R² and R³ radicals are each defined as tert-butyl.

7. The method of claim 1, wherein the palladium catalyst is a homogeneous or heterogeneous catalyst.

8. The method of claim 1, wherein the compounds (B) comprise unsaturated dicarboxylic acids and dicarboxylic acid derivatives having 4 to 10 carbon atoms.

9. The method of claim 1, wherein the reaction is carried out in an aprotic solvent.

10. The method of claim 1, wherein the reaction is carried out in the absence of a solvent.

11. The method of claim 1, wherein the reaction is carried out in the absence of acids having a pKa of 3 or less.

12. The method of claim 1, wherein the reaction is carried out in the absence of oxygen.

13. The method of claim 1, wherein the compounds (A) comprise a compound selected from the group consisting of the unsaturated carboxylic acids having 14 to 24 carbon atoms and the esters of unsaturated carboxylic acids having 14 to 24 carbon atoms.

14. The method of claim 1, wherein the reaction is carried out at a temperature of about 40 to about 80° C.
